(19) United States
(12) Patent Application Publication
(10) Pub. No.: US 2008/0045981 A1
(43) Pub. Date: Feb. 21, 2008

(54) LIGATING CLIP AND LIGATING CLIP APPLICATOR

(76) Inventors: Ilya Margolin, Longmeadow, MA (US); Stanley Kowalski III, Wilbraham, MA (US)

Correspondence Address: LAW OFFICE OF MICHAEL A. BLAKE, 112 BROAD STREET, MILFORD, CT, 06460, US

(21) Appl. No.: 11/379,821

(22) Filed: Apr. 24, 2006

Related U.S. Application Data
(60) Provisional application No. 60/673,853, filed on Apr. 22, 2005.

(51) Int. Cl. A61B 17/08; A61B 17/10

(52) U.S. Cl. 606/151; 606/139; 606/142

(57) ABSTRACT

A ligating clip comprising: a first member; a connecting member in communication with the first member; a second member in communication with the connecting member; a first generally orthogonal semicircular member in communication with the first member; a second generally orthogonal semicircular member in communication with the second member; a third member in communication with the first generally orthogonal semicircular member; a fourth member in communication with the second generally orthogonal semicircular member, and adjoining the third member; a planar arcual member in communication with both the third member and the fourth member; a rectangular opening defined by the first member, the second member, the connecting member, and portions of the first and second generally orthogonal semicircular members, and where the opening has an opening width; an adjoined width defined by the adjoined width of the third member and the fourth member; and wherein the adjoined width is greater than the opening width. A ligating clip applicator comprising: an upper handle; a lower jaw member in fixed communication with the upper handle; a lower handle in rotative communication with the upper handle; an upper jaw member in fixed communication with the lower handle; an offset upper jaw member in fixed communication with the upper jaw member; an offset lower jaw member in fixed communication with the lower jaw member; a clip housing in fixed communication with the upper jaw, the clip housing configured to hold a plurality of ligating clips; an advancing rod in operational communication with the clip housing, and configured to advance ligating clips located in the clip in a direction generally from the housing towards; a translating spring block in fixed communication with the advancing rod; a stationary block guide in slideable communication with the advancing rod, and in fixed communication with the lower handle; a stationary spring block in fixed communication with the stationary block guide; and a tension spring in communication with the stationary spring block and the translating spring block.

### CROSS-REFERENCES

This patent application claims the benefit of provisional patent application Ser. No. 60/673,853 by Ilya Margolin and Stanley Kowalski, entitled “Surgical clips and reloadable multi-clip applier”, filed on Apr. 22, 2005, the entire contents of which are fully incorporated by reference herein.

### TECHNICAL FIELD

The present invention relates generally to surgical instruments and methods, and more specifically to devices and methods for ligation, surgical wound closure, tissue approximation and attachment, and vascular anastomosis.

### BACKGROUND

Ligation of veins, arteries, other blood vessels, and ducts is common in many surgical procedures. Referring to FIG. 1, conventional ligation clips 10 are typically made of titanium wire and are U-shaped with two extending legs 14, 18 that are bent inwardly by an applicator 22. The jaws 26 of the applicator 22 close together to crimp the clip 10 on a blood vessel. Clips are intended to remain in place after ligation. However, because of distending (inflation of the vein), softness, slipperiness of blood vessels and influence of surrounding body muscles and other organs some clips can become dislodged or displaced over period of time. It is believed that up to 30% of clips are displaced. It is common practice to install multiple clips, up to four or five clips, to make a more reliable ligation of one blood vessel. As an example, 100-200 clips are considered an average usage for one coronary artery bypass graft (CABG).

Existing clip applicators are known to contain complex mechanisms in order to feed and crimp a clip. FIG. 2 shows an exploded view of a typical surgical clip applicator 22. The applicator in FIG. 2 represents a standard repeating multi-clip applicator. Due to the complexity and intricate nature of the applicator it is very difficult to sterilize the vast number of confined spaces in the applicator. Therefore the existing applicators are designed and accepted to be limited in multiple use or disposable. The complexity of the mechanism is necessary to properly contain and automatically feed a series of prior art flat u-shaped ligating clips.

Additionally, the forming of the clip is done by use of a scissor like mechanism attached to a sliding mechanism ultimately resulting in the forming of the u-shaped clip. The feeding of the clip in a prior art applicator 22 is performed during the first stage of squeezing the handle and the forming is done during the second stage of squeezing. This requires extra travel of the handle as compared to the manual applicators 30, one of which is shown in FIG. 3. The added travel of the handle results in fatigue and repetitive motion injuries. Additionally, the complex mechanism results in the isolation of tactile feedback from the clip forming tip. This lack of feedback results in uncertainty by the surgeon as to whether the clip has been successfully advanced and formed. Manual applicators 30, such as the one shown in FIG. 3, are preferred for their tactile feedback. However the applicators 30 are configured to install one clip at a time, and do not allow for the quick and accurate use of a large number of clips that may be required for a procedure.

Therefore a clip and clip applicator that overcomes the disadvantages described above as well as other disadvantages are needed.

### SUMMARY

The disclosed invention relates to a ligating clip comprising: a first member; a connecting member in communication with the first member; a second member in communication with the connecting member; a first generally orthogonal semicircular member in communication with the first member; a second generally orthogonal semicircular member in communication with the second member; a third member in communication with the first generally orthogonal semicircular member; a fourth member in communication with the second generally orthogonal semicircular member, and adjoining the third member; a planar arcual member in communication with both the third member and the fourth member; a rectangular opening defined by the first member, the second member, the connecting member, and portions of the first and second generally orthogonal semicircular members, and where the opening has an opening width; an adjoined width defined by the adjoined width of the third member and the fourth member; and wherein the adjoined width is greater than the opening width.

The disclosed invention also relates to a ligating clip applicator comprising: an upper handle; a lower jaw member in fixed communication with the upper handle; a lower handle in rotative communication with the upper handle; an upper jaw member in fixed communication with the lower handle; an offset upper jaw member in fixed communication with the upper jaw member; an offset lower jaw member in fixed communication with the lower jaw member; a clip housing in fixed communication with the upper jaw, the clip housing configured to hold a plurality of ligating clips; an advancing rod in operational communication with the clip housing, and configured to advance ligating clips located in the clip in a direction generally from the housing towards; a translating spring block in fixed communication with the advancing rod; a stationary block guide in slideable communication with the advancing rod, and in fixed communication with the lower handle; a stationary spring block in fixed communication with the stationary block guide; and a tension spring in communication with the stationary spring block and the translating spring block.

### BRIEF DESCRIPTION OF THE DRAWINGS

The present disclosure will be better understood by those skilled in the pertinent art by referencing the accompanying drawings, where like elements are numbered alike in the several figures, in which:

FIG. 1 is a front view of a prior art clip applicator and ligating clip;

FIG. 2 is an exploded view of a prior art ligating clip applicator;

FIG. 3 is a front view of a prior art manual applicator;

FIG. 4 is a perspective view of a disclosed ligating clip;

FIG. 5 is a perspective view of the disclosed ligating clip adjacent to a blood vessel;

FIG. 6 is rear view of the view in FIG. 5;

FIG. 7 is a perspective view of the disclosed ligating clip partially closed on a blood vessel;

FIG. 8 is a cross-sectional view of the disclosed ligating clip partially closed on a blood vessel;

FIG. 9 is a cross-sectional view of the disclosed ligating clip fully closed on a blood vessel;

FIG. 10 is a perspective view of a disclosed ligating clip applicator;

FIG. 11 is a front view of the disclosed ligating clip applicator from FIG. 10;

FIG. 12 is a top view of the disclosed ligating clip applicator from FIG. 10;

FIG. 13 is a cross-sectional view of the disclosed ligating clip applicator;

FIG. 14 is a cross-sectional view of the disclosed ligating clip applicator with the advancing rod removed; and

FIG. 15 is a cross-sectional view of the disclosed ligating clip applicator showing a series of ligating clips located in the clip housing.

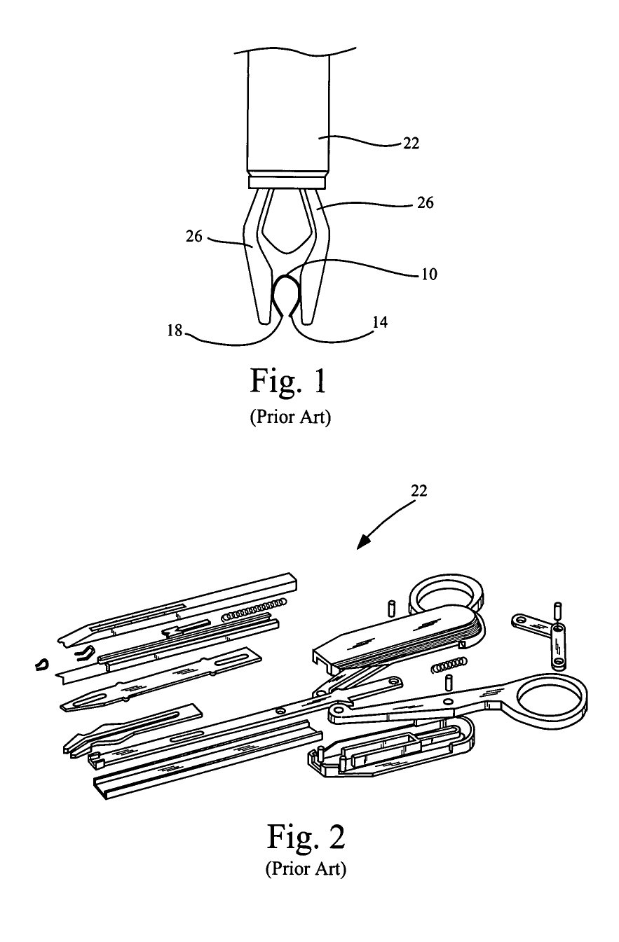

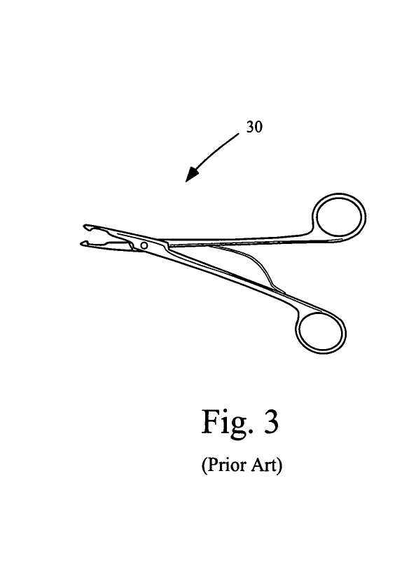

### DETAILED DESCRIPTION

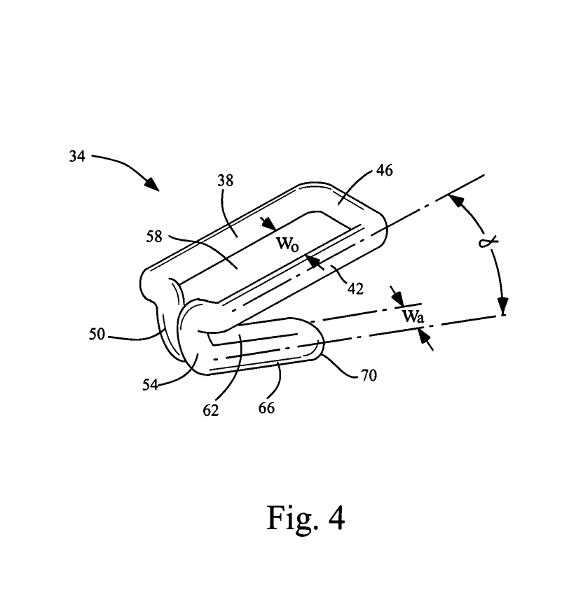

FIG. 4 is a perspective view of a disclosed ligating clip 34 in an “open” orientation. The ligating clip 34 comprise a first member 38, a second member 42 and a connecting member 46. The first member 38, second member 42, connecting member 46 generally lie in a first plane. The first member 38 is in communication with a first generally orthogonal semicircular member 50, and the second member 42 is in communication with a second generally orthogonal semicircular member 54. The first member 38, second member 42, connecting member 46 and portions of the first and second generally orthogonal semicircular members 50, 54 form a generally rectangular opening 58. The first generally orthogonal semicircular member 50 is in communication with a third member 62. The second generally orthogonal semicircular member 54 is in communication with a fourth member 66. A planar arcual member 70 is in communication with both the third member 62 and fourth member 66. The planar arcual member 70, the third member 62, and fourth member 66 all generally lie in a second plane. The angle between the first and second planes, shown by \acute{α}, is about 18° to about 35°, and preferably about 20°. The third member 62 and fourth member 66 are generally parallel and adjoining to one another. The rectangular opening has an opening width Wₒ of a certain value. The adjoined width Wₐ of the third member 62 and fourth member 66 is of a certain value. Wₒ is generally smaller than Wₐ. The diameter of the clip material is about 0.02 inches to about 0.05 inches (about 0.5 mm to about 1.2 mm). The clips 34 may be made out of any suitable material for ligating clips, including but not limited to: titanium, nitinol, stainless steel.

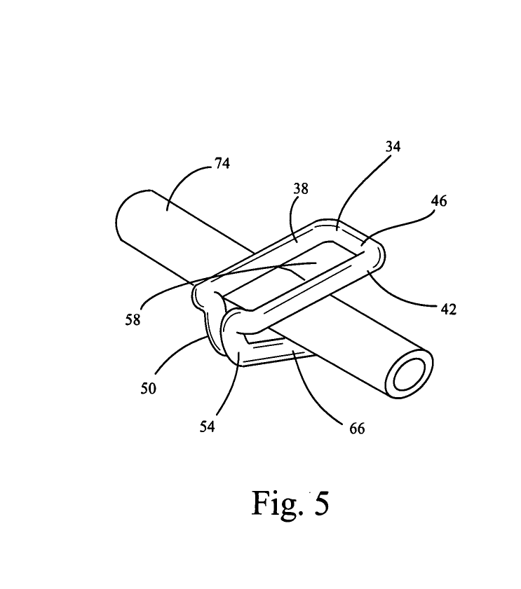

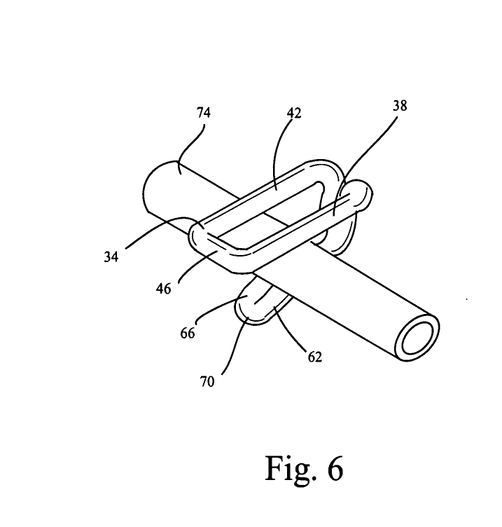

FIG. 5 is a perspective view of the ligating clip 34 that is about to be used on a blood vessel 74. FIG. 6 is a reverse perspective view of FIG. 5. Upon installation of the ligating clip 34, the ligating clip 34 is positioned so that the blood vessel 74 is generally centered between the first member 38, second member 42, and connecting member 46 on the one hand, and the third member 62, fourth member 66, and planar arcual member 70 on the other hand.

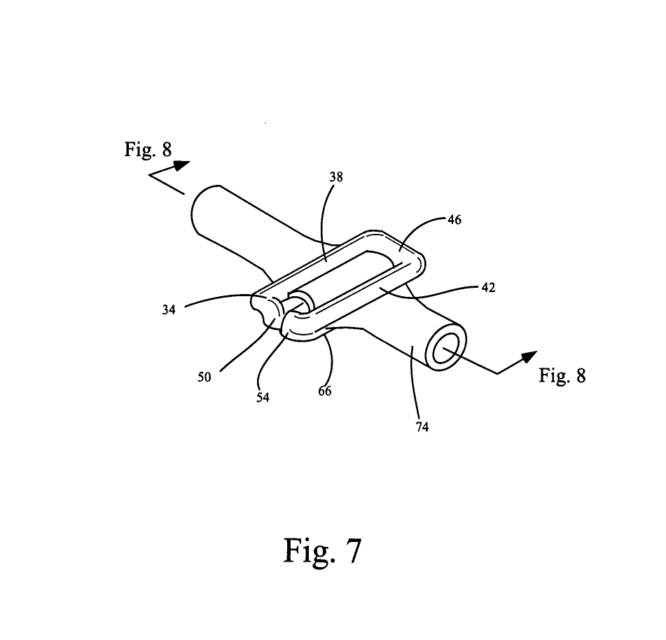

FIG. 7 is a perspective view of the ligating clip 34 that has been installed on a blood vessel 74. Because Wₒ is generally smaller than Wₐ, there is an interference fit between the third, fourth, and arcual members 62, 66, 70, and the opening 58 (now filled by the blood vessel 74, and the third, fourth, and arcual members 62, 66, 70).

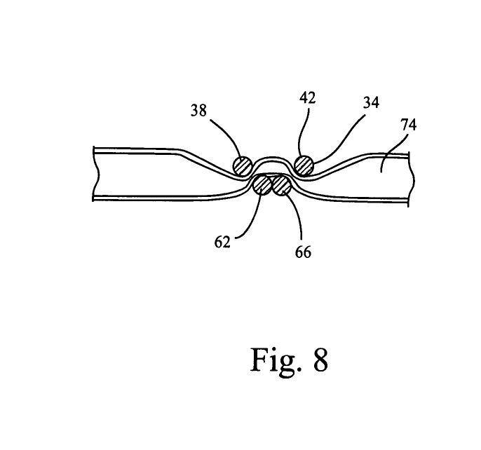

FIG. 8 shows a cross-sectional view of the ligating clip 34 and blood vessel 74. The clip 34 is shown in a partially closed orientation in this view. Even partially closed, it can be seen that the blood flow through the blood vessel 74 will be either completely stopped or greatly reduced. In this partially closed orientation, the first and second members 38, 42 are slightly offset from third and fourth members 62, 66 (i.e. they are non-coplanar).

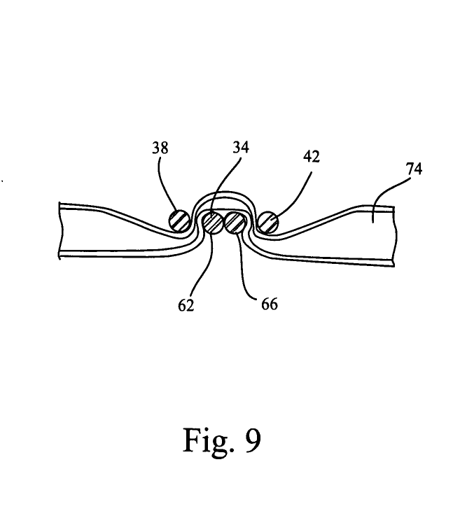

FIG. 9 shows a cross-sectional view of the ligating clip 34 and blood vessel 74, but with the clip 34 fully closed, i.e. the first and second members 38, 42 are generally coplanar with the third and fourth members 62, 66. In other words, the first plane is coplanar with the second plane. In this fully closed position, the flow through the blood vessel 74 is completely stopped.

The shape of the ligating clip 34 provides superior retaining force when in an installed or clamped configuration. The ligating clip 34 provides multiple pinch and wrap points on the blood vessel 74. Because the ligating clip 34 has multiple wrap points on the blood vessel 74, there is much less likelihood that the clip 34 will slide off the blood vessel, even if the blood vessel 74 is harvested for a medical procedure such as CABG. The use of the disclosed clip 34 is believed to be as secure as using two or more of the prior art ligating clips on the same blood vessel 74. By using fewer clips 34, a surgeon will experience less fatigue during installation of the clips while still providing a superior clamp/ligation. Additionally, using the disclosed ligating clip 34 will prevent crushing or cutting of the blood vessel 74.

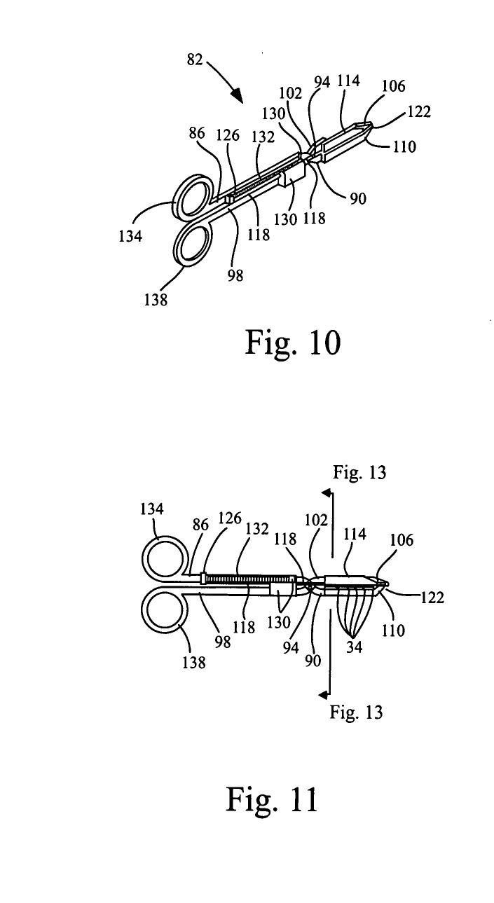

FIG. 10 is a perspective view of a disclosed ligating clip applicator 82. The applicator 82 comprises an upper handle 86 that transitions into a lower jaw 90. The point of transition is generally the pivot point 94 of the applicator. The applicator also comprises a lower handle 98 that transitions into an upper jaw 102. The transition point, is generally the pivot point 94. In communication with the upper jaw 102 is an offset upper jaw member 106. Similarly, in communication with the lower jaw 90 is an offset lower jaw member 110. The lower handle 98, upper handle 86, lower jaw 90, and upper jaw 102 all generally lie in a third plane, the offset upper jaw member 106 and the offset lower jaw member 110 generally lie in a fourth plane parallel to the third plane. A clip housing 114 is in communication with the upper jaw 102. An advancing rod 118 is configured to advance clips located within the clip housing to the installation end 122 of the applicator 82 where the jaw member 106, 110 can be used to compress the clip 34 about a blood vessel. Force is exerted on the advancing rod 118 towards the installation end 122 by a translating spring block 126. The advancing rod 118 is guided by the stationary guide block 130 which is fixed to the lower handle 98. A stationary spring block 130 is fixedly attached to the stationary guide block 130. A tension spring 132 is in communication with the stationary spring block 130 and the translation spring block 126. The translating spring block 126 is allowed to move relative to the upper handle 86. The upper handle 86 may also comprise an upper finger holder 134, and similarly the lower handle 98 may comprise a lower finger holder 138. It should be noted that a person of ordinary skill in art may differently configure the disclosed applicator 82, and such configurations are encompassed by this patent document. The applicator 82 may be made out of any suitable applicator material including, but not limited to: stainless steel, or steel and other alloy with medical grade protective coating. The length of the applicator may be about 8 inches to about 14 inches. The offset upper and lower jaw members 106, 110 open and close with generally a scissor-like action of the applicator 82.

FIG. 11 is a front view of the disclosed applicator 82. In this view a plurality of clips 34 can be seen in the clip housing 114.

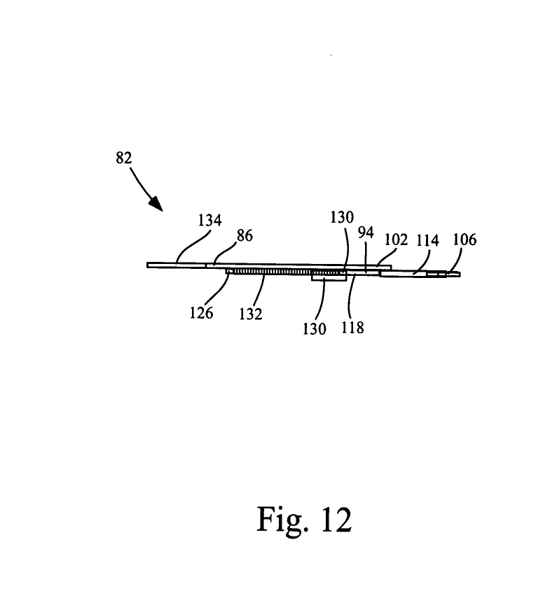

FIG. 12 is a top view of the disclosed applicator 82.

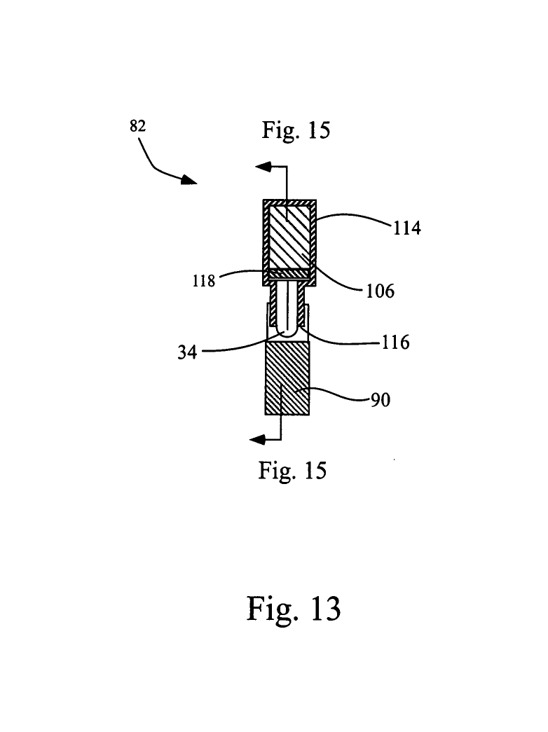

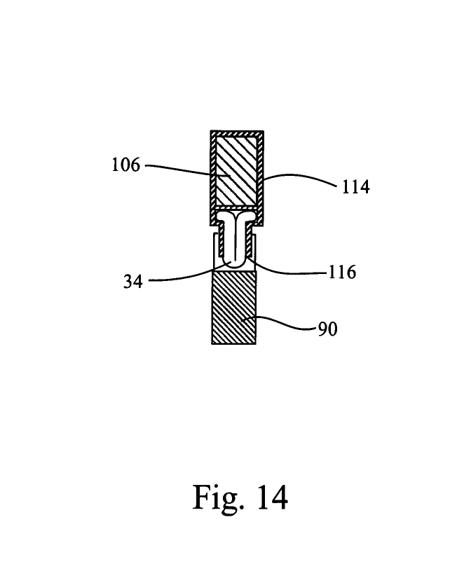

FIG. 13 is a cross-sectional view of the applicator 82 (plane A-A is shown in FIG. 11). FIG. 14 is a cross-sectional view of the applicator 82, with the advancing rod 118 removed so that the clip orientation in the clip housing 114. In this view, it can be seen that the clip housing 114 has a channel 116 (which is filled by the clip 34) in which the clips 34 can slide through (in a direction in and out of the paper).

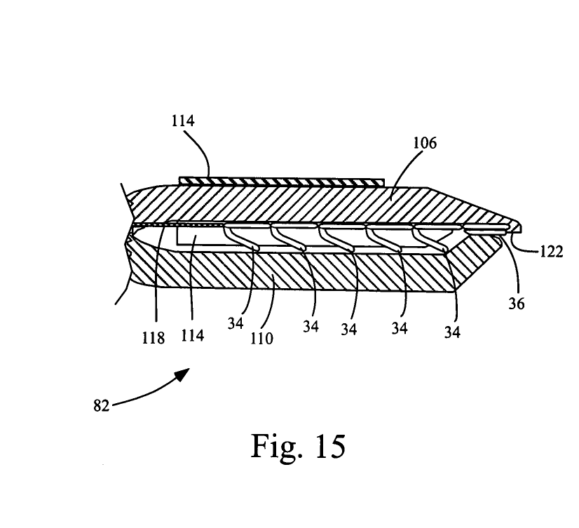

FIG. 15 is a cross-sectional view of the applicator 82 through plane B-B. In this view a plurality of clips 34 are shown in the clip housing 114. The advancing rod 118 is shown in communication with the clip the furthest away from the installation end 122. Clip 36 is shown as it is pressed into a partially closed configuration, by offset upper jaw member 106 and lower offset jaw member 110, so that it is in a partially closed configuration like the clip in FIG. 8. When more force is applied to the applicator, 82, the clip 36 may be pressed into a fully closed configuration like the clip shown in FIG. 9.

The disclosed shape of the ligating clip 34 facilitates the automatic loading and queuing of the clips 34 in the disclosed applicator 82. The clip housing 114 allows for a rail style loading of the clip 34. This rail style loading allows the clips 34 to be positioned end to end. About 5 to about 20 clips 34 may fit in the applicator 82 at one time.

The disclosed clip applicator 82 can house a wide variety of ligating clips and automatically feed those clips toward the offset upper jaw member 106 and lower offset jaw member 110. The clips are aligned in a guidance channel 116 formed in the clip housing 114. A spring loaded advancing rod 118 maintains force on the queue of clips 34, such that when one clip 34 exits the applicator 82, a subsequent clip 34 is pushed towards the installation end 122. Once the clip 34 is installed with respect to a blood vessel, the offset upper jaw member 106 and lower offset jaw member 110 are opened and the next clip from guidance channel 116 moves into offset upper jaw member 106 and lower offset jaw member 110. The rail load method eliminates numerous parts compared to the existing repeating ligating clip applicators. Fewer parts results in less cost to manufacture and lower cost.

Because the disclosed ligating clip applicator has so few parts, sterilization of the applicator 82 is possible. Therefore, the applicator 82 may be used numerous times. Additionally, the clips 34 may be stored in a magazine which allows for a quick load during a medical or surgical procedure.

The clip 34 and applicator 82 disclosed herein are not the totality of embodiments encompassed by this patent document. Other embodiments encompassed by this patent document include numerous configurations of rail type guidance system and multiple pinch points exerted by the clip 34. For example, the clip may be manufactured with open ends or they may be lightly attached to one another with a break away point. Additionally, the applicator 82 may have numerous configurations to allow automatic feeding, for example, a trigger mechanism may be included in the applicator 82 to allow feeding of the queued clip only when the handle is opened to its full extent.

It should be noted that the terms “first”, “second”, and “third”, and the like may be used herein to modify elements performing similar and/or analogous functions. These modifiers do not imply a spatial, sequential, or hierarchical order to the modified elements unless specifically stated.

While the disclosure has been described with reference to several embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but that the disclosure will include all embodiments falling within the scope of the appended claims.

What is claimed is:

1. A ligating clip comprising:
a first member;
a connecting member in communication with the first member;
a second member in communication with the connecting member;
a first generally orthogonal semicircular member in communication with the first member;
a second generally orthogonal semicircular member in communication with the second member;
a third member in communication with the first generally orthogonal semicircular member;
a fourth member in communication with the second generally orthogonal semicircular member, and adjoining the third member;
a planar arcual member in communication with both the third member and the fourth member;
a rectangular opening defined by the first member, the second member, the connecting member, and portions of the first and second generally orthogonal semicircular members, and where the opening has an opening width;
an adjoined width defined by the adjoined width of the third member and the fourth member; and
wherein the adjoined width is greater than the opening width.

2. The ligating clip of claim 1, further comprising:
a first plane defined by the first member, the second member, and the connecting member;
second plane defined by the third member, the fourth member, and the planar arcual member; and
wherein the angle between the first plane and the second plane is about 18° to about 35° when in an opened configuration.

3. The ligating clip of claim 1, wherein the clip is made out of the material selected from the group comprising: titanium, nitinol, stainless steel.

4. The ligating clip of claim 2, wherein when the ligating clip is fully closed the first plane is coplanar with the second plane.

5. The ligating clip of claim 2, wherein when the ligating clip is partially closed, the first plane is generally parallel to the second plane, and the first plane is generally not coplanar with the second plane.

6. The ligating clip of claim 1, wherein when the ligating clip is closed, there is an interference fit of the third and fourth members in the rectangular opening.

7. A ligating clip applicator comprising:
an upper handle;
a lower jaw member in fixed communication with the upper handle;
a lower handle in rotative communication with the upper handle;
an upper jaw member in fixed communication with the lower handle;
an offset upper jaw member in fixed communication with the upper jaw member;
an offset lower jaw member in fixed communication with the lower jaw member;
a clip housing in fixed communication with the upper jaw, the clip housing configured to hold a plurality of ligating clips;
an advancing rod in operational communication with the clip housing, and configured to advance ligating clips located in the clip in a direction generally from the housing towards;
a translating spring block in fixed communication with the advancing rod;
a stationary block guide in slideable communication with the advancing rod, and in fixed communication with the lower handle;
a stationary spring block in fixed communication with the stationary block guide; and
a tension spring in communication with the stationary spring block and the translating spring block.

8. The ligating clip applicator of claim 7, wherein the clip housing has a guidance channel configured to guide ligating clips.

9. The ligating clip applicator of claim 7, wherein the clip housing is configured to hold about 5 to about 20 ligating clips.

10. The ligating clip applicator of claim 7, wherein:
the upper handle, the lower jaw member, the lower handle, and the upper jaw member generally lie in a first plane;
the offset upper jaw member and the offset lower jaw member generally lie in a second plane that is parallel to the first plane, and not coplanar to the first plane.
